SEQUENCE LISTING (21 sequences)

SEQ ID NO: 1 - DNA, 29 bp, homo sapiens
tcctggtacctcctgaaagatgaagctac

SEQ ID NO: 2 - DNA, 33 bp, homo sapiens
ctcaagcttgaattcaataccggcgatacagtc

SEQ ID NO: 3 - DNA, 54 bp, homo sapiens
tcttggtaccatgggccctaaaaagaagcgtaaagtcgccccccgaccgatgtc

SEQ ID NO: 4 - DNA, 31 bp, homo sapiens
tcttgaattccccaccgtactcgtcaattcc

SEQ ID NO: 5 - DNA, 28 bp, homo sapiens
ctctagatctattaccgccatgcattag

SEQ ID NO: 6 - DNA, 19 bp, homo sapiens
ccacaactagaatgcagtg

SEQ ID NO: 7 - DNA, 45 bp, homo sapiens
cctactagtatgggctgtgtgcaatgtaaggataaagaagcagcg

SEQ ID NO: 8 - DNA, 45 bp, homo sapiens
tctgaattcctctgtcagtttcgctgcttctttatccttacattg

SEQ ID NO: 9 - DNA, 25 bp, homo sapiens
tctgaattctatgactgcaaagatg

SEQ ID NO: 10 - DNA, 26 bp, homo sapiens
cctggtacctgcagccgcgggtgctg

SEQ ID NO: 11 - DNA, 22 bp, homo sapiens
tctgaattcccaggagcggatc

SEQ ID NO: 12 - DNA, 26 bp, homo sapiens
cttggtaccaaaaatgtttgcaactg

SEQ ID NO: 13 - DNA, 26 bp, homo sapiens
ctgaattctctccagaagaagaagag

SEQ ID NO: 14 - DNA, 27 bp, homo sapiens
ctactcgaggaaggcctcctcagactc

SEQ ID NO: 15 - DNA, 18 bp, Artificial Sequence
  PCR primer
tcgcgatgtacgggccag

SEQ ID NO: 16 - DNA, 39 bp, Artificial Sequence
  PCR primer
gaattccccaccgtactcgtcctctgtcagtttcgctgc

SEQ ID NO: 17 - DNA, 21 bp, Artificial Sequence
  PCR primer
tggttctttccgcctcagaag

SEQ ID NO: 18 - DNA, 27 bp, homo sapiens
ctcagatctatgtcccaggagcggatc

SEQ ID NO: 19 - DNA, 26 bp, homo sapiens
cttggtaccaaaaatgtttgcaactg

SEQ ID NO: 20 - DNA, 25 bp, homo sapiens
ctcagatctatgactgcaaagatgg

SEQ ID NO: 21 - DNA, 26 bp, homo sapiens
cctggtacctgcagccgcgggtgctg

(19) United States
(12) Patent Application Publication
(10) Pub. No.: US 2003/0211523 A1
(43) Pub. Date: Nov. 13, 2003

(54) Two-hybrid double screening system and method

(76) Inventors: Shuang Zhang, New York, NY (US); Edward B. Ziff, New York, NY (US); Jun Zhong, Somerset, NJ (US)

Correspondence Address: KLAUBER & JACKSON, 411 HACKENSACK AVENUE, HACKENSACK, NJ, 07601

(21) Appl. No.: 10/354,931

(22) Filed: Jan. 30, 2003

Related U.S. Application Data
(60) Provisional application No. 60/353,590, filed on Feb. 1, 2002.

(51) Int. Cl. C12Q 1/68; G01N 33/53; G01N 33/567; C12N 1/18

(52) U.S. Cl. 435/6; 435/7.2; 435/254.2

(57) ABSTRACT

A method of identifying proteins able to interact via protein-protein interactions with a protein of interest, comprising (a) performing a first assay to identify cells expressing a reporter gene, wherein the first assay is transcription-based two-hybrid screening system; (b) obtaining DNA from the cells identified in step (a) via a PCR procedure; and (c) performing a second assay on the DNA obtained in step (b), wherein the second assay is a non-transcription-based two-hybrid screening system, to identify cells expressing a reporter gene, wherein positive cells identified in step (a) that are found to be positive in step (c) express a protein able to interact via protein-protein interactions with a protein of interest.

### CROSS-REFERENCE TO RELATED APPLICATIONS

This Application claims priority to provisional application Serial No. 60/353,590, filed Feb. 1, 2002, incorporated herein by reference in its entirety.

### FIELD OF THE INVENTION

This invention relates to methods of identifying protein-protein interactions comprising a first assay linked to a second assay by PCR.

### BACKGROUND OF THE INVENTION

Protein-protein interactions mediate many important cellular processes and are central to the mechanisms by which most proteins function. For a protein of unknown function, identification of binding partners is greatly helpful in determining its function. In addition, detected phenotypes in many diseases result from dysfunction biomolecular interactions, especially protein-protein interactions. Thus, identifying these aberrant biomolecular interactions allows subsequent identification of compounds able to affect them, thus providing a significant opportunity in the development of disease treatment.

One approach for elucidating protein-protein interactions in cells is the yeast-based two-hybrid system (U.S. Pat. No. 5,283,173), which was originally described more than a decade ago by Fields and Song (1989) Nature 340:245. The system is based on the ability to split a transcription factor into two separable functional domains: a DNA-binding domain (DBD) and a transcriptional activation domain (AD). Each individual domain is unable to activate transcription of a reporter gene on its own. In a two-hybrid system, these two domains are distinct polypeptides fused to a polypeptide. The basis of the assay is that expression of a reporter gene will occur only if X and Y interact to bring the separated DNA-binding domain and the transcriptional activation domain together. Typically, the first hybrid protein, termed “the bait”, consists of a Gal4 DNA-binding domain (Gal4BD) fused to a polypeptide sequence of a known protein. The second hybrid proteins consists of the Gal4 activation domain fused to a polypeptide sequence of a second protein, termed “the prey”. Binding between the two-hybrid proteins reconstitutes the Gal4 DNA-binding domain with the Gal4 activation domain, which leads to the transciptional activation of a reporter gene, such as lacZ) or HIS3) which is operably linked to a Gal4 binding site.

Mammalian two-hybrid systems have been developed, including the Mammalian 2-H Assay Kit (Stratagene), the CheckMate 2-H System (Promega), the Mammalian MATCHMAKER 2-H Assay Kit (Clontech. A more recently described mammalian two-hybrid system is described by Shioda et al. (2000) Proc. Natl. Acad. Sci. USA 97:5220-5224 and U.S. Pat. No. 6,251,676.

### SUMMARY OF THE INVENTION

The invention provides a two-hybrid double screening method which combines a transcription-based two-hybrid screening with a non-transcription-based two hybrid screening, each of which two-hybrid screening is linked by an inter-procedure in which proteins identified as positive in the first screening and directly re-screened in the second screening.

Accordingly, in a first aspect, the invention features a method for identifying proteins able to interact via protein-protein interactions with a protein of interest, comprising: (a) performing a first assay to identify cells expressing a reporter gene, wherein the first assay is transcription-based two-hybrid screening assay; (b) obtaining DNA from the cells identified in step (a) via a PCR procedure; and (c) performing a second assay on the DNA obtained in step (b), wherein the second assay is a non-transcription-based two-hybrid screening assay, to identify cells expressing a reporter gene, wherein positive cells identified in step (a) and confirmed in step (c) express a protein able to interact via protein-protein interactions with a protein of interest.

In one embodiment of the method, the transcription-based assay is a mammalian two-hybrid system. In another embodiment, the transcription-based assay is a yeast two-hybrid system.

In one embodiment, the non-transcription-based two-hybrid system is an in vivo binding screening comprising (a) expressing in a cell a first nucleic acid molecule encoding a first fusion protein comprising a reporter protein fused to a target protein, (b) expressing in the cell a second nucleic acid molecule encoding a second fusion protein comprising a cell membrane localization domain fused to a second protein, and (c) determining the presence of a reporter protein in a membrane of the cell, wherein the presence of a reporter protein in the membrane indicates a protein-protein interaction between the target protein and the second protein.

In one embodiment, the first screening assay and/or the second screening assay is conducted for 30 hours or less. In a more specific embodiment, the first screening assay and/or the second screening assay is conducted for 25 hours or less. In still more specific embodiments, the first screening assay and/or the second screening assay is conducted for 20 hours or less, or 15 hours or less.

In another specific embodiment, positive cells in the first screening assay are identified by high-speed fluorescence activated cell sorting (FACS).

In a second aspect, the invention features a kit for detecting protein-protein interactions, comprising:

(a) first screening assay components, comprising a first mammalian cell comprising (i) a reporter gene encoding a reporter polypeptide operably linked to an upstream transcriptional regulatory sequence comprising a DNA binding site for a DNA binding domain, (ii) a bait nucleotide sequence encoding a bait fusion protein, the bait fusion protein comprising a DNA binding domain and the bait; and (iii) a cDNA library of prey, wherein each prey cDNA encodes a prey fusion protein comprising a transcriptional activation domain and a prey;

(b) 3-primer PCR components; and

(c) second screening assay components, comprising a second mammalian cell, the second mammalian cell comprising (i) a bait nucleotide sequence encoding the bait fusion protein, wherein the bait fusion protein comprises a reporter polypeptide and a bait protein, (ii) a cDNA prey PCR product; wherein the first screening identifies a candidate prey cDNA encoding a prey able to interact with the bait, and the 3-primer PCR results in a PCR product comprising the candidate prey cDNA and a membrane-localizing signal, and wherein the second screening identifies a prey cDNA encoding a prey able to interact with the bait.

In one embodiment, the first screening assay and/or the second screening assay is conducted for 30 hours or less. In another embodiment, the reporter gene and transcriptional regulatory sequence in the first mammalian cell is integrated into a chromosome of the first mammalian cell. In further embodiments, the DNA binding domain is the Gal4 DNA binding domain and the DNA binding site is Gal4 DNA binding site, and the transcriptional activation domain is VP16. In a further embodiment, the reporter polypeptide is a fluorescent polypeptide; in a more specific embodiment, the fluorescent polypeptide is green fluorescent protein (GFP).

In a third aspect, the invention features a method for detecting protein-protein interactions, comprising:

(a) conducting a first screening assay with a first mammalian cell, wherein the first mammalian cell comprises (i) a reporter gene encoding a reporter polypeptide operably linked to an upstream transcriptional regulatory sequence, wherein the transcriptional regulatory sequence comprises a DNA binding site, (ii) a bait nucleotide sequence encoding a bait fusion protein, wherein the bait fusion protein comprises a DNA binding domain and the bait; and (iii) a cDNA library of prey, wherein each prey cDNA encodes a prey fusion protein comprising a transcriptional activation domain and a prey, wherein the first mammalian cell is incubated under conditions conducive to expressing the reporter gene in the presence of an interaction between a bait protein and a prey protein;

(b) performing a 3-primer PCR resulting in a PCR product comprising the candidate prey cDNA and a membrane-localizing signal; and

(c) conducting a second screening assay with a second mammalian cell, the second mammalian cell comprising (i) a bait nucleotide sequence encoding the bait fusion protein, wherein the bait fusion protein comprises a reporter polypeptide and a bait protein, and (c) the cDNA prey PCR product of step (b), and wherein the second screening identifies a prey cDNA encoding a prey able to interact with the bait.

In a specific embodiment, the first mammalian cell comprises a reporter gene encoding a fluorescent polypeptide operably linked to an upstream transcriptional regulatory sequence comprising a DNA binding site for a DNA-binding domain, the reporter gene and transcriptional regulatory sequence being integrated into a chromosome of the mammalian cell.

In another specific embodiment, the first mammalian cell comprises a DNA molecule comprising (i) a first nucleotide sequence encoding a bait fusion protein comprising an upstream Gal4 DNA-binding domain and the bait; (ii) OriP; and (iii) a nucleotide sequence encoding Epstein-Barr virus nuclear antigen 1 (EBNA-1) protein. In another embodiment, the first mammalian cell comprises a DNA molecule comprising (i) a detectable marker; (ii) a nucleotide sequence encoding prey fusion protein comprising an upstream VP16 transcriptional activation domain and the prey; and (iii) OriP.

In a fourth aspect, the invention features a method for screening interactions between a bait and a library of prey in a mammalian cell, comprising:

(a) providing a first mammalian cell comprising (i) a reporter gene encoding a fluorescent polypeptide operably linked to a transcriptional regulatory sequence containing a DNA binding site for a DNA-binding domain, (ii) a bait nucleotide sequence encoding a bait fusion protein, the bait fusion protein comprising a DNA binding domain and the bait; and (iii) a cDNA library of prey, wherein each prey cDNA encodes a prey fusion protein including a transcriptional activation domain and a prey;

(b) incubating the mammalian cell for 30 hours or less;

(c) detecting expression of the reporter gene, if present, wherein the detecting is collecting signal positive cells;

(d) rescuing cDNA from the recovered positive cells of step (d) with 3-primer PCR, wherein rescued cDNA comprises a cDNA prey PCR product comprising a membrane-localizing signal and a prey;

(e) providing the cDNA prey PCR product in a second mammalian cell, the second mammalian cell further comprising a bait nucleotide sequence encoding the bait fusion protein, wherein the bait fusion protein comprises a fluorescent polypeptide and a bait protein;

(f) incubating the second mammalian cell for 30 hours or less;

(g) detecting membrane-bound signal of the fluorescent polypeptide, if present, wherein detection of a membrane-bound fluorescent signal indicates an interaction between the bait and the prey.

FIG. 1 shows one specific embodiment of the method of the invention.

In a fifth aspect, the invention features a method for reducing false positives obtained in a conventional yeast two-hybrid screening, comprising:

(a) isolating prey cDNAs from yeast colonies resulted from the yeast two-hybrid screening;

(b) performing a 3-primers PCR procedure to prepare the prey cDNAs in an expressible form in order to be re-examined in the second screening;

(c) performing a second screening comprising the steps of: (i) providing a mammalian cell comprising: (1) a bait nucleotide sequence encoding the bait fusion protein, comprising a fluorescent polypeptide and a bait; and (2) a cDNA prey PCR product, comprising a membrane-localizing signal and a prey, resulted from the 3-primers PCR;

(d) incubating the mammalian cell for 30 hours or less; and

(e) detecting membrane-bound signal of the fluorescent polypeptide, if present, indicating an interaction between the bait and the prey.

In a sixth aspect, the invention features a method of confirming positive results obtained in a conventional yeast two-hybrid screening, comprising:

(a) isolating prey cDNAs from yeast colonies resulted from the yeast two-hybrid screening;

(b) performing a 3-primers PCR procedure to prepare the prey cDNAs in an expressible form in order to be re-examined in the second screening;

(c) performing a second screening comprising the steps of: (i) providing a mammalian cell comprising: (1) a bait nucleotide sequence encoding the bait fusion protein, comprising a fluorescent polypeptide and a bait; and (2) a cDNA prey PCR product, comprising a membrane-localizing signal and a prey, resulted from the 3-primers PCR;

(d) incubating the mammalian cell for 30 hours or less; and

(e) detecting membrane-bound signal of the fluorescent polypeptide, if present, indicating an interaction between the bait and the prey.

In a seventh aspect, the invention features a method for detecting an interaction between a bait and a prey in a mammalian cell, comprising:

(a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey;

(b) incubating the cell for 30 hours or less; and

(c) detecting the location of the fluorescent polypeptide signal, if present surrounding the cell membrane, indicating an interaction between the bait and the prey.

In a eighth aspect, the invention features a method of identifying an agent that disrupts interaction between a bait and a prey, comprising:

(a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey;

(b) contacting the mammalian cell with a test agent;

(c) incubating the cell for 30 hours or less; and

(d) detecting a decrease of the fluorescent polypeptide signal surrounding the cell membrane compared to the level of the fluorescent polypeptide signal surrounding the cell membrane in the absent of the test agent, if present, thereby detecting an agent that disrupts an interaction between the bait and the prey.

In an ninth aspect, invention further features a method of identifying an agent able to enhance interaction between a bait and a prey, including: (a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey; (b) contacting the mammalian cell with a test agent; (c) incubating the cell for 30 hours or less; and (d) detecting a increase of the fluorescent polypeptide signal surrounding the cell membrane compared to the level of the fluorescent polypeptide signal surrounding the cell membrane in the absent of the test agent, if present, thereby detecting an agent able to enhance an interaction between the bait and the prey.

Other features and advantages of the invention will be apparent from the detailed description, the drawings, and the claims.

### BRIEF DESCRIPTION OF THE DRAWINGS

FIG. 1 is a diagram of the one embodiment of the method of the invention including a mammalian two-hybrid double screening system.

FIG. 2 is a diagram of prey vector PMFYN and bait vectors PMNGFP and PMCGFP useful in one embodiment of the first two-hybrid screening.

FIG. 3 is a diagram of vectors useful in one embodiment of an in vivo binding screening of the method of the invention.

FIG. 4 is a diagram of the general principle of the first two-hybrid screening step of the invention.

FIG. 5 is a diagram of a 3-primer PCR procedure.

FIG. 6 is a diagram of an in vivo binding screening.

FIG. 7 is a diagram the general procedure of the yeast two-hybrid double screening system useful in the method of the invention.

### DETAILED DESCRIPTION

Before the present assay methodology, and treatment methodology are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Definitions

The term “different detecting principles” is used herein to mean that detected phenotypes are resulted from different biological mechanisms, e.g., transcription-based phenotypes utilized by conventional two-hybrid systems, cdc25-2 rescued phenotype utilized by the protein recruitment system (SRS), and GFP re-localization phenotype utilized by the in vivo assay described herein.

The term “screening system” is used herein to mean that a system use a defined protein to search for nucleotides encoding its binding proteins among a cDNA library. The defined protein is used as a bait fusion protein while a cDNA library is constructed in prey fusion proteins. It will be appreciated by those skilled in the art that many variations of the prey and bait fusion proteins can be constructed and is considered within the scope of the present invention. For example, it will be understood that, for screening polypeptide libraries, the library can be cloned into either the bait or prey fusion proteins. In this sense, the terms “prey” and “bait” are merely convenient names for fusion proteins with transcriptional activation domains and DNA-binding domains, respectively.

The term “detection system” is used herein to mean that a system could be used to determine whether two defined testing proteins interact or not. One test protein is used in a “bait” fusion protein, while the other is used in a “prey” fusion protein.

General Description

An important challenge faced with the use of prior art yeast two-hybrid systems is the identification and removal of false positives from the screening, in part resulting from detection of protein-protein interaction by an indirect phenotype. Many factors contribute to the appearance of false positives, and a large investment of time and labor is required to eliminate these false positive.

To overcome the weakness of the yeast two-hybrid system, a mammalian two-hybrid system is desirable. There are many different types of mammalian cells, so that proper conditions can be obtained to identify protein-protein interactions. In addition, mammalian proteins are less likely to be toxic, and thus better tolerated in mammalian cells than in yeast. Several attempts have been made to apply the principle of the yeast two-hybrid system to mammalian cells, although, generally many currently available mammalian two-hybrid systems are relatively inefficient, and useful only as a detection system to confirm interactions between two testing proteins, not as a screening system to search for new interaction partners using a known protein bait. Among commercially available mammalian two-hybrid systems are Mammalian 2-H Assay Kit (Stratagene), CheckMate 2-H System (Promega), Mammalian MATCHMAKER 2-H Assay Kit (Clontech), etc.

Another mammalian two-hybrid system is described recently in U.S. Pat. No. 6,251,676. In this system, CV-1yEBNA-1 monkey kidney epithelial cells expressing Epstein-Barr virus nuclear antigen 1 (EBNA-1) were stably transfected with a reporter plasmid for Gal4-dependent expression of the green fluorescent protein (GFP). A resulting clone, GB133, could maintain plasmids containing the OriP Epstein-Barr virus replication origin that directs replication of plasmids in mammalian cells in the presence of the EBNA-1 protein. When the bait-expressing GB133 cells were transfected transiently with an OriP-containing expression plasmid for the positive prey, the cells were readily identified by the green fluorescence in cell culture and eventually formed green fluorescent microcolonies. The green fluorescent microcolonies were harvested directly from the culture dishes under a fluorescence microscope. Prey-coding cDNA was recovered by PCR using primers annealing to the vector sequences flanking the insert-cloning sites.

There are several drawbacks in using the system described in U.S. Pat. No. 6,251,676 to search for protein-protein interactions by using a bait. Since up to 10⁸ transfectants need to be screened in order to cover the entire mammalian genome, it is extremely difficult to search for GFP-positive cells using a fluorescent microscope and isolate them by conventional cell cloning methods using small plastic cylinders, as described in U.S. Pat. No. 6,251,676. Secondly, it does not address the issue of false positives coming out from the screening. It is expected that more false positives will result in the screening of a mammalian cDNA library compare to a yeast library because of the larger size of the mammalian library; thus how to separate real interactions from false positives in an efficient way is indispensable for a mammalian two-hybrid screening system.

The two-hybrid double screening system described herein is designed to accelerate identifying authentic protein-protein interactions and distinguishing these from the false positives generated in both prior art yeast and mammalian two-hybrid systems. The instant method also provides modifications and improvements over existing mammalian two-hybrid systems such that the methods provided herein are more suitable for identifying protein-protein interaction screening in a mammalian two-hybrid double screening system. In addition, the second screening in this system may be used alone as a mammalian in vivo system for detecting interactions between two testing proteins.

The invention provides a method comprising two independent two-hybrid systems together to carry out protein-protein interaction screening in a mammalian/yeast cell. One of the advantages of the method presented herein, is that since the two screenings are based on different detecting principles, the second screening functions as an efficient way to remove false positives, as well as an independent way to confirm the real protein-protein interactions. It saves a considerable amount of time, resources and labor relative to the combination of a traditional two-hybrid screenings with an in vitro binding examination, which is the procedure in common usage.

Further, the second screening used in the method of the invention is a direct examination of the relationship between two proteins, as described below, thus any interaction identified by this system is very compelling. Still further, both screenings are carried out in vivo, no in vitro binding conditions is required to be configured experimentally.

In specific embodiments, the method of the invention includes a 3-primer PCR procedure, described below, which rescues cDNAs encoding potential interactors from the first screening and prepares them for direct examination in the second screening. The 3-primer PCR step connects both screenings tightly and facilitates the rapid identification of authentic protein-protein interactions.

The term “transcription-based phenotypes” is used herein to mean that two types of hybrid proteins are prepared. The first hybrid contains the DNA-binding domain of a transcriptional activator fused to the first test protein. The second hybrid protein contains a transcriptional activation domain fused to the second test protein. If the two test proteins are able to interact, they bring into close proximity the two domains of the transcriptional activator. This proximity is sufficient to cause transcription, which can be detected by the activity of a marker gene that contains a binding site for the DNA-binding domain. A nonexclusive list of commonly used reporter or marker genes include LacZ, HIS3, LEU2, ADE2, and URA3. Thus, the principle of detecting a protein-protein interaction in a transcription-based assay is detecting reporter gene expression.

“Cdc25-2 rescued phenotype” is a method that detects a protein-protein interaction by the recruitment of an effector protein, which is not a transcription factor, to a particular cell compartment, where the effector protein can activate a reporter molecule. The protein recruitment system is exemplified by a protein-protein interaction that results in recruitment of a guanine nucleotide exchange factor to the plasma membrane, where it effects the activation of a Ras reporter molecule. Interaction between the bait and expression of the prey recruits hSos to cell membrane. Membrane localizing hSos protein fully suppressed the growth deficiency of cdc25-2 mutants at 36° C., a temperature at which they normally undergo G1 arrest due to an inability to activate Ras. Thus, the principle of detecting a protein-protein interaction with the cdc25-2 rescued assay is detecting the ability of cdc25-2 cell to grow at high temperature.

A “GFP re-localization phenotype” means that a protein-protein interaction is detected by the recruitment of a GFP protein to a particular cell compartment. Since the reporter, GFP, is fused with a bait protein, the distribution of GFP in cells directly reflects the interaction between the bait and the prey. Thus, the principle of detecting a protein-protein interaction with the GFJP relocalization assay is detecting membrane-bound GFP localization.

Two-Hybrid Double Screen System

The invention provides a method by combining two two-hybrid systems together, which systems are based on different detecting principles, to carry out protein-protein interaction screening in a mammalian/yeast cell. This method allows protein-protein interactions identified in the first screening to be directly confirmed in the second screening. The instant invention thus largely eliminates false positives initially identified. The second screening step is also a direct examination of protein-protein interaction, and thus does not rely on indirect phenotypes used in other two-hybrid screenings. One advantage of the method of the invention is that both screening are conducted in vivo, and thus in vitro binding conditions do not need to be first determined experimentally. The in vivo binding system described herein may be used as part of the two-hybrid double screening method of the invention, or alone used as a detection system to determine protein-protein interaction between two testing proteins.

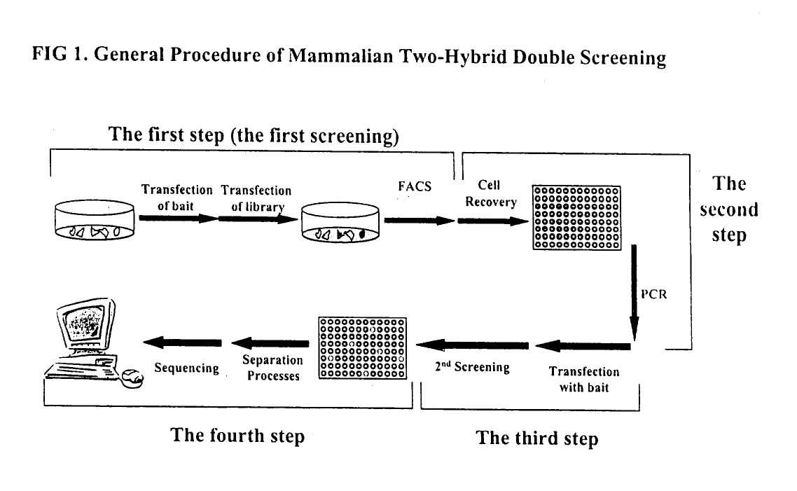

One embodiment of a mammalian two-hybrid double screening system method of the invention includes is shown in FIG. 1. Cells are transfected with a bait construct and a cDNA library simultaneously or sequentially. After FACS screening, cells expressing high level of GFP will be collected in 96-well plates and recovered to form colonies. cDNA is rescued by 3-primers PCR procedure and transfected into cells with the bait-GFP construct for an in vivo binding screening. A cDNA identified as positive in the first screening and reconfirmed positive in the second screening is isolated and sequenced.

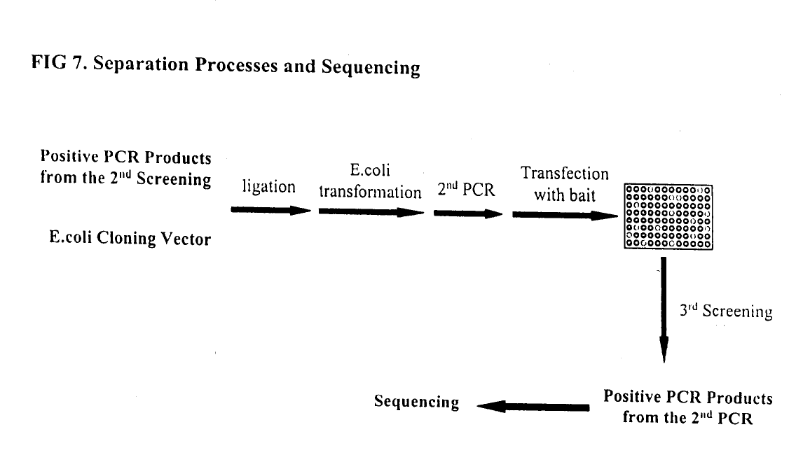

In another embodiment, a yeast two-hybrid double screening method is used to identify protein-protein interaction. FIG. 7 shows an embodiment in which a traditional yeast two-hybrid screening is performed as the first screening assay. After isolating prey DNA from yeast colonies, prey DNA for an in vivo binding screening (second screening assay) is prepared by a 3-primers PCR procedure. Then the PCR product is transfected directly into mammalian cells with the bait-GFP construct for the second two-hybrid screening. A PCR product confirmed as positive in the second screening step is sequenced.

Transcription-Based Two-Hybrid Screening Assay

The first screening assay of the method of the invention is to identify cells containing candidate proteins potentially able to interact with the protein of interest, and to collect the positive cells (and ultimately the cDNAs encoding the candidate interacting proteins). The first screening assay of the invention is a transcription-based two-hybrid system, which allows even weak and transient interactions to be detected as signal accumulated over certain time.

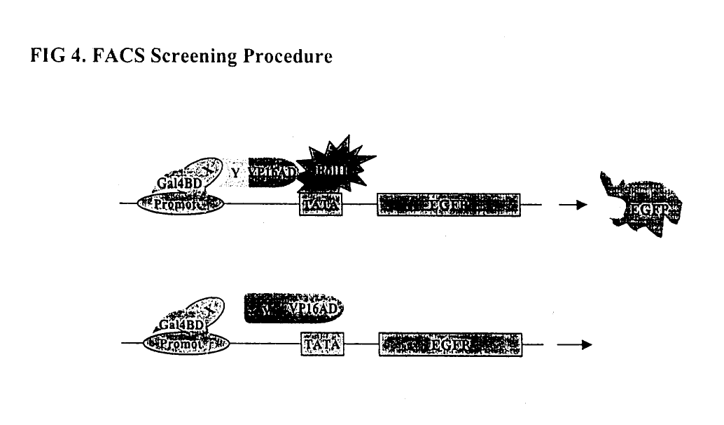

One embodiment of the first two-hybrid screening step of the invention is shown in FIG. 4. In this embodiment, X is the bait fused with Gal4 binding domain (Gal4BD) and Y is cDNA library fused with VP16 activation domain (VP16AD). An interaction between X and Y will bring Gal4BD and VP16AD together to promote GFP expression, which is detected by FACS.

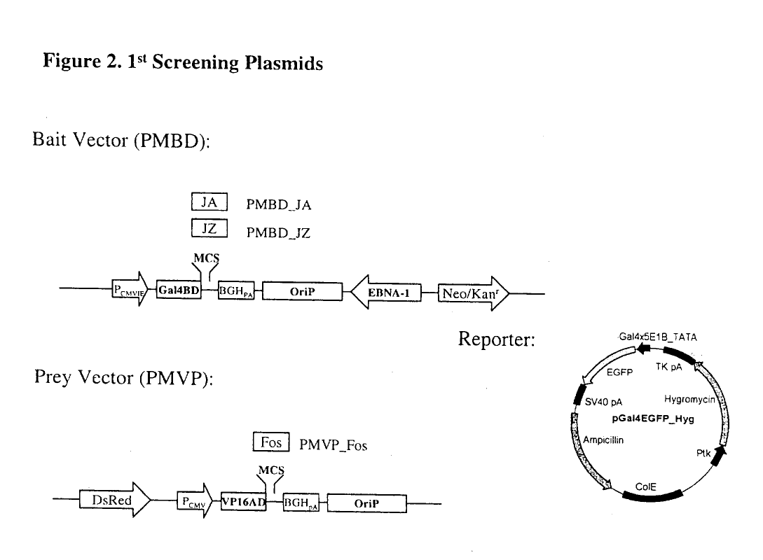

Another embodiment of a first two-hybrid screening is shown in FIG. 2. In this embodiment, bait DNA is cloned 3′ to Gal4-binding domain in plasmid PMBD. The cDNA library is constructed downstream of VP16 activation domain in plasmid PMVP. Both PMBD and PMVP have OriP so that they are able to be maintained in cells expressing EBNA-1. In addition, PMBD has genes encoding EBNA-1 and neomycin resistant protein. PMVP has a DsRed gene. pGal4EGFP_Hyg is the reporter including GFP driven by a Gal4 minimal promoter. Cells with the reporter integrated may be selected with hygromycin in cell media.

In one embodiment of the two-hybrid double screening system of the invention, the first screening is known in the art, U.S. Pat. No. 6,251,676. However, several modifications may be introduced to improve the suitability of that assay for use in the instant invention:

GFP expressing cells are sorted by FACS. Typically, multiple GFP-positive cells (usually 5-10 cells) are collected together into 96-well plates. The use of cell sorting by FACS provides improved speed and sensitivity. Cell sorting by FACS may be performed at a very rapid rate (up to 70,000 cells/second). Therefore, 10⁷-10⁸ cells, enough to cover the entire genome, can be easily screened in a very short time, e.g., less than one hour. Further, FACS is very sensitive to the GFP signal. Cells expressing GFP at a level of several folds above background could be easily detected and sorted. Another advantage to the FACS for cell sorting is increased flexibility. For example, the threshold above which cells are collected can be varied depending on the background signal and stringency of selection, which provides better results than the use of growth vs. no growth phenotype used in yeast two-hybrid systems.

Use of a detectable marker in the prey vector. A red fluorescent polypeptide may be used to estimate the number of transfectants screened in FACS.

OriP present on both bait and prey vectors, and EBNA-1 is expressed on the bait vector. By including the EBNA-1 sequence in the vectors, this system can be used in a broader range of cell lines, and is not limited to a cell line which expresses the EBNA-1 protein. Therefore, cells which do not express EBNA-1 endogenously, may also be used in this screening system.

Other transcription-based mammalian two-hybrid systems may be used as in the first screening assay. For example, OriP and EBNA-1 may be omitted from the bait and the prey constructs if prey cDNAs are recovered by RT-PCR from potential interaction-positive cells. In this case, the sorted GFP-positive cells are used directly in a RT-PCR procedure to rescue cDNAs into an expressible form, which may be directly re-examined in the in vivo binding system.

In the yeast two-hybrid double screening system, a useful first screening assay may be that described in U.S. Pat. No. 5,283,173. However, other transcription-based yeast two-hybrid systems may be used as a first screening assay.

Preparation of Prey cDNA Libraries

As described herein, a mammalian/yeast two-hybrid double screening system can be used to screen for protein-protein interactions with a know protein bait against a prey cDNA library. Exemplary cDNA libraries could be prepared from many sources, such as animals, plants, fungus and/or microbes. cDNA libraries can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Recovery of Positive Cells and 3-primer PCR

Cells identified as positive in the first screening method are recovered into colonies, and their prey cDNAs rescued into an expressible form which is directly re-examined in the second screening.

In GFP-positive (prey) cells are detected in the mammalian two-hybrid double screening system, the cells are collected into, for example, 96-well plates by FACS. Cells recover and form colonies in about 5-7 days. Methods for improving cell growth may be used, such as, for example, minimizing time in FACS buffer on ice, use of conditional media, coated plates, and adding the present of untransfected cells.

In one embodiment of the invention, total DNA prepared from recovered cell colonies is used as template for 3-primer PCR. In another embodiment, prey cDNA is rescued by (i) preparing DNA from cell colonies, (ii) transforming E. coli with the DNA prepared in step (i); (iii) isolating DNA from several E. coli colonies; and (iv) using the isolated DNA as template to perform 3-primer PCR.

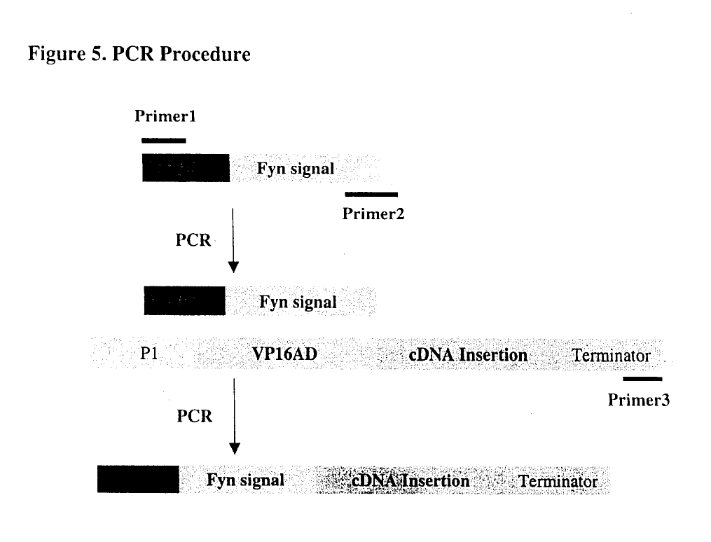

3-primer PCR procedure: 3-primer PCR is performed using prey cDNAs as templates. The PCR product, shown in FIG. 5, is a linear DNA fragment containing a promoter region, a start codon (ATG), a membrane-localizing signal in frame with a prey followed by a transcription termination region.

The 3-primer PCR procedure is conducted as follows: (a) first and second primers are used to prime synthesis from the rescued prey cDNA to produce a DNA fragment containing (i) a promoter region, (ii) a translation start codon (ATG), (iii) a membrane-localizing signal, and (iv) a region which is overlapping with the upstream region of the prey cDNA in the transcription-based two-hybrid system; (b) the DNA fragment generated in step (a) pairs with a third primer to produce a DNA fragment which may be used directly as the prey DNA construct in the second screening assay (in vivo binding screening) described below. This prey DNA construct consists of (i) a promoter region, (ii) a translation start codon (ATG), (iii) a membrane-localized signal, (iv) a prey DNA sequence, and (v) a transcription terminator.

Alternatively, the 3-primer PCR procedure may be conducted by (a) using primers 2 and 3 to produce a DNA fragment containing a region overlapping with (i) a 3′ of a promoter region, (ii) a start codon (ATG), (iii) a membrane-localizing signal, (iv) a prey DNA sequence, and (v) a transcription terminator in the transcription-based two-hybrid system; (b) the DNA fragment generated in step (a) is paired with primer 1 to produce a DNA fragment directly usable as the prey construct in the subsequent in vivo binding screening. This prey construct consists of (i) a promoter region, (ii) a translation start codon (ATG), (iii) a membrane-localizing signal, (iv) a prey DNA, and (v) a transcription terminator.

In a yeast two-hybrid double screening system, prey cDNAs are rescued from yeast colonies by methods well known to the art. 3-primer PCR is then performed using prey cDNAs as templates. The PCR product is a linear DNA fragment containing (i) a promoter region, (ii) a membrane-localizing signal in frame with a prey DNA sequence, and (iii) a transcription termination region. The PCR product may be directly re-examined in the second screening.

Non-Transcription Based (In Vivo Binding) Assay

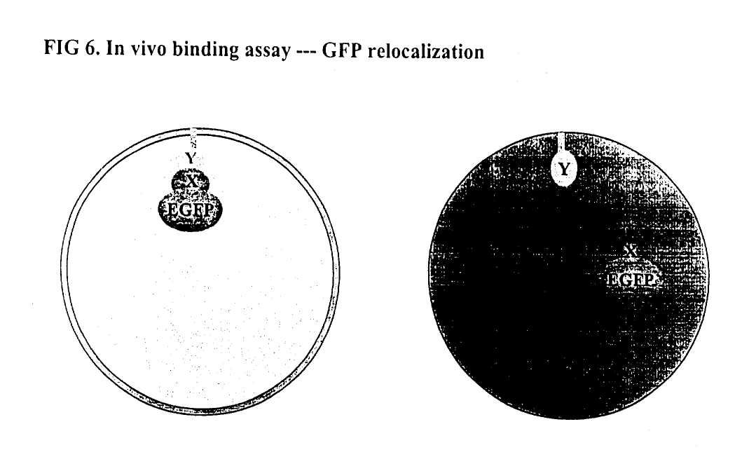

In part, the second screening functions to remove false positives generated in the first screening. The second screening is an in vivo binding system, for example, as shown in FIG. 6. In one embodiment, X is the bait fused with GFP and diffusely distributed in cytoplasm when expressed alone. Y is a cDNA fused with a membrane-localizing signal. Only upon interaction between X and Y, cells display membrane-bound GFP signal.

Among the advantages provided by the in vivo binding system assay of the method of the invention are (1) extremely low background, since this assay is not dependent on any indirect phenotypes used in the yeast SRS system and the conventional two-hybrid system; (2) easy detection. One disadvantage of this step of the method, however, is that currently there are no machines available that can distinguish membrane-localizing signals from cytoplasm-localizing signals. Since the first screening limits the number of cDNAs to be examined in the second screening, it is feasible to use the in vivo binding system as the second screening. Moreover, since the first and the second screenings are based on different principles, each interaction identified in the method of the invention has been confirmed by two independent approaches.

The in vivo binding system described herein can be used for a variety of different purposes, e.g., for screening agents which can act as agonists or antagonists of protein-protein interactions. In a general sense, the assay evaluates the ability of an agent to modulate (e.g., enhance or inhibit) binding between the bait and prey polypeptides. Exemplary agents include peptides, nucleic acids, carbohydrates, small organic molecules, and natural product extract libraries, such as isolated from animals, plants, fungus and/or microbes.

More specifically, in one embodiment the in vivo binding system assay is useful to determine if an agent of interest can modulate the binding between a bait and a prey fusion protein known to interact. The ability of a test agent to modulate the interaction can be determined by detecting an increase or decrease in the presence of bait-GFP fusion protein on the cell membrane. A decrease in presence of bait-GFP fusion protein on the cell membrane in the presence of the agent of interest relative to a control (expression of the reporter gene in the absence of the test agent) means that the test agent inhibits the interaction between the bait and prey. Alternatively, when a test agent causes an increase in amount of bait-GFP fusion protein in the cell membrane relative to a control, the test agent is enhances the interaction between the bait and prey polypeptides.

The in vivo binding system described herein may also be used to map residues of a protein involved in a known protein-protein interaction. Thus, for example, various forms of mutagenesis can be utilized to generate a combinatorial library of either bait or prey polypeptides, and the ability of the corresponding fusion protein to bind its partner assayed.

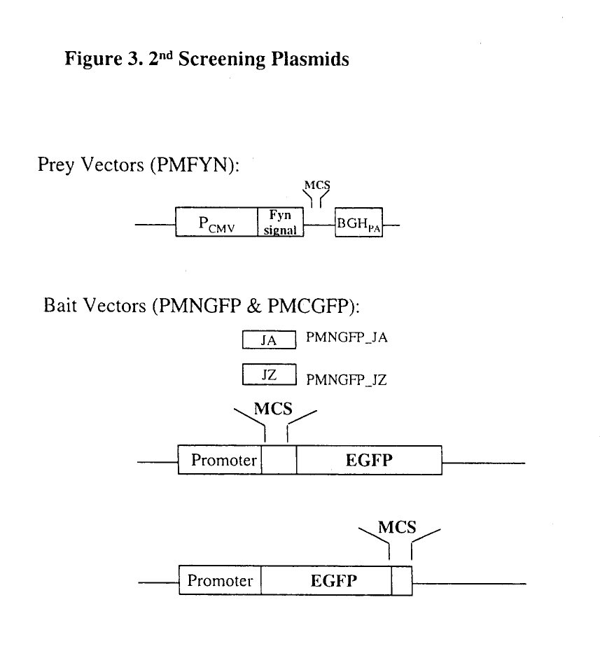

Vectors which may be used in the in vivo binding assay method of the invention are shown in FIG. 3. In one embodiment, bait is cloned 3′ or 5′ to GFP sequence in PMCGFP or PMNGFP, respectively. PMFYN, which contains a Fyn signal under control of a promoter, is used as the template for a 3-primers PCR procedure. A testing protein coding sequence may also be cloned 3′ to Fyn signal in PMFYN.

Bait Fusion Protein

The bait fusion protein used in an in vivo binding system includes a fusion between a polypeptide moiety of interest (e.g., a protein of interest or a polypeptide from a polypeptide library) and a fluorescent polypeptide. The nucleotide sequence which encodes the polypeptide moiety of interest is cloned in-frame to a nucleotide sequence encoding the fluorescent polypeptide.

The bait portion of the bait fusion protein, which is the same bait protein used in the first screening, may be chosen from any protein of interest and includes proteins of unknown, known, or suspected diagnostic, therapeutic, or pharmacological importance.

In one embodiment of the invention, the fluorescent polypeptide is fused in frame to the bait. The advantages of using a fluorescent protein are that: (i) a fluorescent protein generally does not affect the bait; (ii) the signal of a fluorescent protein could be detected as early as 16 hours using an inverted phase-contrast microscope equipped with an epifluorescence light source and a fluorescein isothiocyanate filter set; (iii) positive cells can be identified without damage to the cells.

The bait fusion protein should not display membrane-bounded localization. General speaking, this requirement is easily met, since the same bait is also used in the first screening, which is transcription-based and occurs in the nucleus. It is also desirable that the bait not interfere with the fluorescent polypeptide. Prior to use, the bait fusion protein should be tested to ensure that it is not membrane-bounded. To do so, the construct encoding the bait fusion protein is transfected alone into a mammalian cell and the location of the fluorescent signal is determined under fluorescent microscope.

A preferred fluorescent polypeptide is derived from a GFP. The GFP gene was originally cloned from the jellyfish Aequorea victoria. It encodes a protein of 238 amino acids which absorbs blue light (major peak at 395 nm) and emits green light (major peak at 509 nm) (Prasher et al. (1992) Gene 15:229-223). GFP genes and functional proteins have been identified in a variety of organisms in the phyla hydrozoa, cnidaria, anthozoa and ctenophora. Both wild-type GFP and mutated GFP from Aequorea victoria may be used in the instant invention. The mutation of GFP (e.g., the substitution of certain amino acids in the GFP polypeptide) has been reported to yield GFP proteins with improved spectral properties. For example, mutating serine 65 to a threonine generates a GFP variant which has about six fold greater brightness than wild-type GFP (Heim et al. (1995) Nature 372:663-664). The coding sequence for an enhanced GFP can be purchased commercially (Clontech, Palo Alto, Calif.). In some embodiments, a mammalian-optimized version of a GFP cDNA is used.

Blue fluorescent protein (BFP) may also be used as a reporter gene in the method of the invention. To obtain BFP, tyrosine 66 of GFP is mutated to a histidine. This mutated GFP protein fluoresces bright blue, in contrast to the green of the wild-type protein. Other variants of GFP include yellow fluorescent protein (YFP), and cyan fluorescent protein (CFP). Other suitable fluorescent proteins include those described by Matz et al. (1999) Nature Biotechnology 17:969-973.

DsRed and its variant, a series of red fluorescent proteins, can also be used. DsRed1 and DsRed2 are derived from the Discosoma sp. red fluorescent protein (drFP583; 1). DsRed2, like its progenitor DsRed1, contains a series of silent base-pair changes that correspond to human codon-usage preferences for high expression in mammalian cells. In addition to these changes, DsRed2 contains six amino acid substitutions: A105V, I161T, and S197A, which result in the more rapid appearance of red fluorescence in transfected cell lines; and R2A, K5E, and K9T, which prevent the protein from aggregating.

HrGFP (Stratagene, La Jolla, Calif.), a green fluorescent protein isolated from Renilla reniformis, can also be used. HrGFP has characteristics that make it a superior alternative to the Aequorea protein for use as a biological marker. Renilla hrGFP absorbs light with a 2.5-fold higher extinction coefficient than EGFP, has a broader range of pH stability than Aequorea GFP, and has low toxicity mammalian expression.

Prey Fusion Protein

The prey fusion protein in an in vivo binding system includes a membrane-localizing signal and a candidate interactor polypeptide sequence which is tested for the ability to form an intermolecular association with the bait polypeptide. In the method of the invention, the prey fusion protein localizes on the cell membrane, and protein-protein contact between the bait and prey fusion proteins (via the interaction of the bait and prey polypeptide portions of these proteins) recruits the fluorescent protein of the bait fusion protein to the membrane, generating a membrane-bounded signal different from original distribution of the bait fusion protein (FIG. 6).

Any of a number of membrane-localizing signals can be used in the prey fusion protein. For example, the membrane-localizing signal may be: (i) a myristoylation signal that is derived from a number of proteins, including Fyn, Myr, v-Src, etc; (ii) a signal for farnesylation and palmitoylation in many proteins, including H-Ras, etc; (iii) a transmembrane domain in many proteins, including members of the TNF family of proteins, etc.

The term “cell compartment localization domain” is used herein to mean a peptide or polypeptide sequence that directs translocation of a fusion protein containing the sequence to a particular cell compartment. Such domains are referred to herein as “localizing” or “localization” domains, signals or sequences. The term “plasma membrane” is used herein to mean the membrane that delimits a cell, except that it can be contained within a cell wall or cell coat.

In one embodiment of the invention, the membrane-localizing signal is derived from Fyn. As described above, the DNA construct encoding the prey fusion protein is generated by the 3-primer PCR procedure, which generates a linear DNA fragment consisted of a promoter region, a start codon (ATG), the Fyn membrane-localizing signal, the prey cDNA and a transcription termination region.

In another embodiment of the invention, the nucleotide sequence which encodes the prey polypeptide moiety is directly cloned in-frame to a membrane-localizing signal in the prey vector.

Transfection of Cells and Screening

The bait and the prey are co-transfected into a mammalian cell by methods known to the art. After an incubation period of 30 hours or less, cells are examined under a fluorescent microscope. As described above, cells containing the bait alone or containing non-interacting prey proteins display GFP as a diffuse distributing in the cytoplasm. Upon interaction between the bait and the prey, GFP is recruited to the cell membrane, and cells positive for an interacting prey protein display a membrane-bounded GFP distribution. A cell exhibiting the phenotype of GFP localized in the cell membrane is a positive cell, and the corresponding PCR product used to transfect that cell is analyzed further as containing a DNA sequence encoding a protein able to interact with the bait.

Identification of Positive cDNAs

The PCR product prepared from the recovered cell colonies may contain several different cDNA insertions if multiple cells are collected into a single well during FACS, or due to the ability of mammalian cells to take up more than one plasmid during transfection. Therefore, an optional separation step may be used to distinguish cDNAs encoding authentic bait-binding proteins.

In the mammalian two-hybrid double screening system, the following steps may be carried out: (i) positive PCR product from the second screening are inserted into an E. coli vector; (ii) E. coli are transformed with the vector; (iii) E. coli colonies are picked up and used as templates for additional round of PCR with primers 1 and 3 used in the 3-primer PCR procedure; (iv) the PCR products are re-transfected into mammalian cells with the bait-GFP fusion construction used in the second screening; (v) the transfected mammallian cells are incubated for 30 hours or less; (vi) detection of membrane-bound bait-GFP signal, if present, is carried out and indicative of an interaction between the bait and the prey. Positive cDNA clones are sequenced.

In a yeast two-hybrid double screening system, the separation step described above is not required as generally yeast contain only one prey.

Host Cells

Any cultured mammalian cell may be used in the in vivo binding system, for example, a primary, secondary, or immortalized cell. Exemplary mammalian cells are those of mouse, hamster, rat, rabbit, dog, cow, and primate including human. They may be of a wide variety of tissue types, including mast cells, endothelial cells, hepatic cells, kidney cells, or other cell types.

A primary cell is a cell isolated from a mammal (e.g., from a tissue source), which is grown in culture for the first time before subdivision and transfer to a subculture. A secondary cell is a cell at all subsequent steps in culturing. That is, the first time a plated primary cell is removed from the culture substrate and replated (passaged), it is referred to as a secondary cell, as are all cells in subsequent passages. Examples of mammalian primary and secondary cells which can be transfected include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types.

Immortalized cells are cell lines that exhibit an apparently unlimited lifespan in culture. Examples of immortalized human cell lines useful for the present mammalian two-hybrid system include, but are not limited to, HT1080 cells (ATCC CCL 121), HeLa cells and derivatives of HeLa cells (ATCC CCL 2, 2.1 and 2.2), MCF-7 breast cancer cells (ATCC BTH 22), K-562 leukemia cells (ATCC CCL 243), KB carcinoma cells (ATCC CCL 17), 2780AD ovarian carcinoma cells (Van der Blick, A. M. et al., Cancer Res, 48:5927-5932 (1988), Raji cells (ATCC CCL 86), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38VA13 subline 2R4 cells (ATCC CLL 75.1), and MOLT-4 cells (ATCC CRL 1582), as well as heterohybridoma cells produced by fusion of human cells and cells of another species. Secondary human fibroblast strains, such as WI-38 (ATCC CCL 75) and MRC-5 (ATCC CCL 171) may be used. In one embodiment, a HEK-297T cell is used.

Methods of transfecting the DNA molecules described herein (e.g., the reporter gene and associated DNA binding sites, or DNA molecules that encode EBNA-1, the bait fusion protein, or prey fusion protein) into a mammalian cell can be carried out using procedures known in the art. Examples of transfection methods include calcium phosphate or calcium chloride co-precipitation, DEAE-dextran-mediated transfection, lipofection, biolistic transfer, or electroporation. Suitable methods for transfecting host cells in vitro can be found in Sambrook et al., eds., Molecular Cloning: A Laboratory Manual (2nd ed., Cold Spring Harbor Laboratory Press, Cold Spring Harbor, N.Y., 1989) and other laboratory manuals. In one embodiment, cells are transfected using LipofectAMINE PLUS reagent (Gibco/BRL, San Fran., Calif.).

Kits

The invention provides a kit for detecting interaction between two proteins of interest. In an illustrative embodiment, the kit includes at least two constructs. In a specific embodiment, the kit includes: (a) a first gene construct which includes a regulatory sequence operably linked to a nucleotide sequence encoding a fluorescent protein, and a cloning site, (e.g., a convenient cloning site which contains a unique restriction site(s)) for inserting a nucleotide sequence which encodes a bait. The cloning site could be either 5′ or 3′ to the fluorescent protein coding sequence. The nucleotide sequence which encodes the bait is expressed in frame with the fluorescent protein. The kit further includes: (b) a second gene construct including a regulatory sequence operably linked to a nucleotide sequence encoding a myristoylation signal, and a cloning site, (e.g., a convenient cloning site which contains a unique restriction site(s)) for inserting a nucleotide sequence which encodes a prey such that the prey is expressed in frame with the myristoylation signal; and (c) instructions for use.

The kit can also include materials for the mammalian two-hybrid double screening system, which contains: (a) a first gene construct, including an oriP, a sequence encoding EBNA-1, neomycin resistant gene, a regulatory sequence operably linked to a nucleotide sequence encoding a DNA-binding domain, and a cloning site, (e.g., a convenient cloning site which contains a unique restriction site(s)) for inserting a nucleotide sequence which encodes a bait. The nucleotide sequence which encodes the bait is expressed in frame with a DNA-binding domain; (b) a second gene construct including an oriP sequence, a sequence encoding DsRed, a regulatory sequence operably linked to a nucleotide sequence encoding a transcriptional activation domain, and a cloning site, (e.g., a convenient cloning site which contains a unique restriction site(s)) for inserting a nucleotide sequence which encodes a prey such that the prey is expressed in frame with the transcriptional activation domain; (c) a mammalian cell, comprising a reporter gene encoding a fluorescent polypeptide operably linked to a transcriptional regulatory sequence including a DNA binding site for the DNA-binding domain, wherein the reporter gene expresses the fluorescent polypeptide when the bait and prey interact; (d) primers, which can be used in a 3-primers PCR procedure to rescue and amplify the prey sequence, resulted from the first two-hybrid screening, into an expressible form for re-examination using the in vivo binding screening; (e) a third gene construct which includes a regulatory sequence operably linked to a nucleotide sequence encoding a fluorescent protein, and a cloning site, (e.g., a convenient cloning site which contains a unique restriction site(s)) for inserting a nucleotide sequence which encodes a bait. The cloning site could be either 5′ or 3′ to the fluorescent protein coding sequence. The nucleotide sequence which encodes the bait is expressed in frame with the fluorescent protein; (f) a fourth gene construct including a regulatory sequence operably linked to a nucleotide sequence encoding a myristoylation signal, and a cloning site, (e.g., a convenient cloning site which contains a unique restriction site(s)) for inserting a nucleotide sequence which encodes a prey such that the prey is expressed in frame with the myristoylation signal; and (g) instructions for use. Optionally, the kit can also include bacterial cells into which one can introduce total DNA from a mammalian cell that was identified to contain a positive interaction between the bait and prey.

### EXAMPLES

The following examples are put forth so as to provide those of ordinary skill in the art with a complete disclosure and description of how to make and use the two-hybrid double screening method of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1.

Interaction of c-Fos and c-Jun.

A two-hybrid double screening assay was conducted using Gal4 as the transcriptional activator and two interacting human testing proteins, c-Fos and c-Jun. JZ is the c-Jun leucine zipper domain (c-Jun aa 249-331) and binds to a part of c-Fos (c-Fos aa 133-239). JA is the transcriptional activation domain of c-Jun (aa 1-223). JA is able to induce the expression of GFP in the first transcription-based two-hybrid screening when it is fused with a DNA-binding domain even though it does not interact with Fos, since it contains the transcriptional activation domain. Thus, JA is used as a false positive in the first screening. The experiments below demonstrate that the instant methods of the invention are able to identify and eliminate false positives in the second screening.

Plasmid Construction

PMBD: A fragment (EcoR1 digestion and filled-in, then NotI digestion) containing oriP and EBNA-1 from PCEP4 (Invitrogen) is inserted into DsRed-N1 (Clontech) at AflII (filled-in) and NotI. The resulting plasmid was then cut with NheI and XbaI (filled-in) and the fragment containing EBNA-1 and oriP was inserted into pcDNA3.1+ (Invitrogen) at NheI and PvuII to create plasmid DSCE. A PCR fragment, prepared with the oligonucleotides of SEQ ID NO: 1 and SEQ ID NO: 2, containing Gal4BD was inserted into DSCE at EcoR1 and Asp718I to form plasmid PMBD.

PMVP: A fragment (AccI digestion and filled-in) containing oriP from PCEP4 (Invitrogen) was inserted into PcDNA3.1+ at PvuII. The resulting plasmid then had a PCR fragment prepared with the oligonucleotides of SEQ ID NO: 3 and SEQ ID NO: 4, containing VP16AD, inserted at EcoRI and Asp718I to create plasmid pVP16. A PCR fragment, prepared with the oligonucleotides of SEQ ID NO: 5 and SEQ ID NO: 6, containing a red fluorescent polypeptide from DsRed-N1, was inserted into pVP16 at BglII and NruI to form plasmid PMVP.

PMFYN: Two complementary oligonucleotides (SEQ ID NO: 7 and SEQ ID NO: 8) encoding a myristoylation signal in Fyn, were annealed and inserted into PcDNA3.1+ at NheI and EcoRI.

PMBD_JA (FIG. 2): A PCR fragment, prepared with the oligonucleotides of SEQ ID NO: 9 and SEQ ID NO: 10 and containing the JA domain (aa 1-223) of c-Jun, was inserted into PMBD at EcoRI and XhoI (filled-in). The PMBD_JA PCR fragment encodes a protein that is used as a false positive in the first screening.

PMBD_JZ (FIG. 2): A PCR fragment, prepared with the oligonucleotides of SEQ ID NO: 11 and SEQ ID NO: 12 and containing the JZ domain (aa 249-331) of c-Jun, was inserted into PMBD at EcoRI and XhoI (filled-in). The PMBD_JZ PCR fragment encodes a protein that functions as the bait in the first screening.

PMVP_Fos (FIG. 2): A PCR fragment, prepared with the oligonucleotides of SEQ ID NO: 13 and SEQ ID NO: 14 and containing aa 133-239 of c-Fos, was inserted into PMVP at EcoRI and XhoI. The PMVP Fos PCR fragment encodes a protein that functions as the prey in the first screening.

PMNGFP_JA (FIG. 3): A PCR fragment, prepared with the oligonucleotides of SEQ ID NO: 20 and SEQ ID NO: 21 and containing the JA domain of c-Jun, was inserted into pEGFP-N1 (Clontech) at BglII and Asp718I. The PMNGFP_JA PCR fragment encodes a protein which is used as the bait protein in the second screening.

PMNGFP_JZ (FIG. 3): A PCR fragment, prepared with the oligonucleotides of SEQ ID NO: 18 and SEQ ID NO: 19 and containing JA domain of c-Jun, was inserted into pEGFP-N1 at BglII and Asp718I. The PMNGFP_JZ PCR fragment encodes a protein which is used as the bait in the second screening.

The relevant portions of the plasmids used in the first screening test encoding the interacting proteins, were inserted into vectors PMVP and PMBD (vectors illustrated in FIG. 2). The JA and JZ fragments were fused with the Gal4 DNA-Binding domain (BD) and the Fos fragment was fused with the VP16 transcription activation domain (VP) (FIG. 2). Upon interaction of the bait and prey proteins, GFP was produced and cells expressing GFP were detected and sorted by FACS.

The HEK-293T cell line was used to generate a cell line, F6, with the reporter stably integrated. F6 cells alone or transfected with the negative controls (PMBD_JZ/PMVP or PMBD/PMVP_Fos) express very low level of GFP while co-transfection of PMBD_JZ and PMVP_Fos increases the GFP expression dramatically. Cells with different levels of GFP expression are distinguished and separated through FACS. For example, when the threshold is set to isolate cells expressing GFP 30-fold or more above the background, only 1 event or less occurs in 10000 for negative controls (PMVP_Fos/PMBD, PMVP/PMBD_JZ). However, PMVP/PMBD_JA and PMVP_Fos/PMBD_JA could induce expression of GFP due to the transcription activity of JA.

After cell detection and collection into 96-well plates by FACS, 5-7 days are required for the cells to recover and form colonies. In this experiment, cell recover rate was above 50%. Plasmids are maintained as cells divide due to the presence of OriP.

cDNAs were recovered from cell colonies by PCR following a strategy using three primers. The first two primers (SEQ ID NO: 15 and SEQ ID NO: 16) amplify a DNA fragment containing a promoter (P_{CMV}), a membrane-localized signal sequence (MLS) and a sequence overlapping with the region just upstream cDNA insert in the VP vector. This DNA fragment pairs with the third primer (SEQ ID NO: 17) to generate a DNA which consists of P_{CMV}, MLS fused with cDNA, and BGH poly(A) terminator.

The first PCR reaction was carried out as follows: 1 min at 95° C.; 45 sec at 54° C.; 1 min at 72° C.; 30 cycles. The second PCR reaction was carried out as follows: 1 min at 95° C.; 1 min at 53° C.; 2 min at 72° C.; 30 cycles. After purification of the 3-primer PCR products with QIAquick PCR purification kit (Qiagen, Calif.), the PCR products were transfected into HEK293T cells directly, which are able to express the cDNA product fused with a MLS (FIG. 5).

The constructions of the relevant portions of the plasmids used in the second screening test were inserted into vector PMNGFP (illustrated in FIG. 3). The JA and JZ fragments were fused with the DNA fragment encoding GFP. In the case of testers, PMNGFP_JZ becomes membrane-bound only when it is co-expressed with the PCR product encoding the Fyn_Fos peptide. This phenomenon is not observed when PMNGFP_JA is expressed with Fyn_Fos, PMNGFP_JZ is expressed alone, or PMNGFP_JZ is expressed with Fyn_JA. Therefore, the in vivo binding screening was able to rule out false positives resulted from a transcription-based two-hybrid screening, like JA in this example. The results are shown in Table 1 below.

TABLE 1
Two-Hybrid Double Screening Able to Distinguish False Positives

| | | FOS | JA |
|---|---|---|---|
| J | 1^{st} screen assay | + | + |
| Z | 2^{nd} screen assay | + | − |

1. A method for identifying proteins able to interact via protein-protein interactions with a protein of interest, comprising:
(a) performing a first assay to identify cells expressing a reporter gene, wherein the first assay is transcription-based two-hybrid screening system;
(b) obtaining DNA from the cells identified in step (a); and
(c) performing a second assay on the DNA obtained in step (b), wherein the second assay is a non-transcription-based two-hybrid screening system, to identify cells expressing a reporter gene, wherein positive cells identified in step (a) that are found to be positive in step (c) express a protein able to interact via protein-protein interactions with a protein of interest.

2. The method of claim 1, wherein the transcription-based system is a mammalian two-hybrid system.

3. The method of claim 1, wherein the transcription-based system is a yeast two-hybrid system.

4. The method of claim 1, wherein the non-transcription-based two-hybrid system is an in vivo binding screening, comprising:
(a) expressing in a cell a first nucleic acid molecule encoding a first fusion protein comprising a reporter protein fused to a first protein;
(b) expressing in the cell a second nucleic acid molecule encoding a second fusion protein comprising a cell membrane localization domain fused to a second protein; and
(c) determining the presence of a reporter protein in a membrane of the cell, wherein the presence of a reporter protein in the membrane indicates a protein-protein interaction between the first protein and the second protein.

5. The method of claim 1, wherein the first screening assay and/or the second screening assay is conducted for 30 hours or less.

6. The method of claim 5, wherein the first screening assay and/or the second screening assay is conducted for 25 hours or less.

7. The method of claim 6, wherein the first screening assay and/or the second screening assay is conducted for 20 hours or less.

8. The method of claim 1, wherein positive cells in the first screening assay are identified by high-speed fluorescence activated cell sorting (FACS).

9. The method of claim 1, wherein the DNA obtained from positive cells identified in step (a) is subjected to a PCR procedure resulting in a PCR product comprising cDNA of a positive cell and a membrane-localizing signal.

10. A kit for detecting protein-protein interactions, comprising:
(a) a first screening assay, comprising a first mammalian cell comprising (i) a reporter gene encoding a reporter polypeptide operably linked to an upstream transcriptional regulatory sequence comprising a DNA binding site for a DNA binding domain, (ii) a bait nucleotide sequence encoding a bait fusion protein, the bait fusion protein comprising a DNA binding domain and the bait; and (iii) a cDNA library of prey, wherein each prey cDNA encodes a prey fusion protein comprising a transcriptional activation domain and a prey;
(b) components for conducting a 3-primer PCR; and
(c) a second screening assay, comprising a second mammalian cell, the second mammalian cell comprising (i) a bait nucleotide sequence encoding the bait fusion protein, wherein the bait fusion protein comprises a reporter polypeptide and a bait protein, (ii) a cDNA prey PCR product; wherein the first screening identifies a candidate prey cDNA encoding a prey able to interact with the bait, and the 3-primer PCR results in a PCR product comprising the candidate prey cDNA and a membrane-localizing signal, and wherein the second screening identifies a prey cDNA encoding a prey able to interact with the bait.

11. The system of claim 10, wherein the first screening assay and/or the second screening assay is conducted for 30 hours or less.

12. The system of claim 10, wherein the reporter gene and transcriptional regulatory sequence in the first mammalian cell is integrated into a chromosome of the first mammalian cell.

13. The system of claim 10, wherein the DNA binding domain is the Gal4 DNA binding domain, the DNA binding site is the Gal4DNA binding site, and the transcriptional activation domain is VP16.

14. The system of claim 10, wherein the reporter polypeptide is a fluorescent polypeptide.

15. The system of claim 14, wherein the fluorescent polypeptide is green fluorescent protein (GFP).

16. A method for detecting protein-protein interactions, comprising:
(a) conducting a first screening assay with a first mammalian cell, wherein the first mammalian cell comprises (i) a reporter gene encoding a reporter polypeptide operably linked to an upstream transcriptional regulatory sequence, wherein the transcriptional regulatory sequence comprises a DNA binding site, (ii) a bait nucleotide sequence encoding a bait fusion protein, wherein the bait fusion protein comprises a DNA binding domain and the bait; and (iii) a cDNA library of prey, wherein each prey cDNA encodes a prey fusion protein comprising a transcriptional activation domain and a prey, wherein the first mammalian cell is incubated under conditions conducive to expressing the reporter gene in the presence of an interaction between a bait protein and a prey protein;
(b) performing a 3-primer PCR resulting in a PCR product comprising the candidate prey cDNA and a membrane-localizing signal; and
(c) conducting a second screening assay with a second mammalian cell, the second mammalian cell comprising (i) a bait nucleotide sequence encoding the bait fusion protein, wherein the bait fusion protein comprises a reporter polypeptide and a bait protein, and (c) the cDNA prey PCR product of step (b), and wherein the second screening identifies a prey cDNA encoding a prey able to interact with the bait.

17. The method of claim 16, wherein the first mammalian cell comprises a reporter gene encoding a fluorescent polypeptide operably linked to an upstream transcriptional regulatory sequence comprising a DNA binding site for a DNA-binding domain, the reporter gene and transcriptional regulatory sequence being integrated into a chromosome of the mammalian cell.

18. The method of claim 16, wherein the first mammalian cell comprises a DNA molecule comprising (i) a first nucleotide sequence encoding a bait fusion protein comprising an upstream Gal4 DNA-binding domain and the bait; (ii) OriP; and (iii) a nucleotide sequence encoding Epstein-Barr virus nuclear antigen 1 (EBNA-1) protein. In another embodiment, the first mammalian cell comprises a DNA molecule comprising (i) a detectable marker; (ii) a nucleotide sequence encoding prey fusion protein comprising an upstream VP16 transcriptional activation domain and the prey; and (iii) OriP.

19. A method for screening interactions between a bait and a library of prey in a mammalian cell, comprising:
(a) providing a first mammalian cell comprising (i) a reporter gene encoding a fluorescent polypeptide operably linked to a transcriptional regulatory sequence containing a DNA binding site for a DNA-binding domain, (ii) a bait nucleotide sequence encoding a bait fusion protein, the bait fusion protein comprising a DNA binding domain and the bait; and (iii) a cDNA library of prey, wherein each prey cDNA encodes a prey fusion protein including a transcriptional activation domain and a prey;
(b) incubating the mammalian cell for 30 hours or less;
(c) detecting expression of the reporter gene, if present, wherein the detecting is collecting signal positive cells;
(d) rescuing cDNA from the recovered positive cells of step (d) with 3-primer PCR, wherein rescued cDNA comprises a cDNA prey PCR product comprising a membrane-localizing signal and a prey;
(e) providing the cDNA prey PCR product in a second mammalian cell, the second mammalian cell further comprising a bait nucleotide sequence encoding the bait fusion protein, wherein the bait fusion protein comprises a fluorescent polypeptide and a bait protein;
(f) incubating the second mammalian cell for 30 hours or less;
(g) detecting membrane-bound signal of the fluorescent polypeptide, if present, wherein detection of a membrane-bound fluorescent signal indicates an interaction between the bait and the prey.

20. A method for reducing false positives obtained in a conventional yeast two-hybrid screening, comprising:
(a) performing a conventional yeast two-hybrid screening;
(b) isolating prey cDNAs from yeast colonies resulted from the yeast two-hybrid screening;
(c) performing a 3-primers PCR procedure to prepare the prey cDNAs in an expressible form in order to be re-examined in the second screening;
(d) performing a second screening comprising the steps of: (i) providing a mammalian cell comprising: (1) a bait nucleotide sequence encoding the bait fusion protein, comprising a fluorescent polypeptide and a bait; and (2) a cDNA prey PCR product, comprising a membrane-localizing signal and a prey, resulted from the 3-primers PCR;
(e) incubating the mammalian cell for 30 hours or less; and
(f) detecting membrane-bound signal of the fluorescent polypeptide, if present, indicating an interaction between the bait and the prey.

21. A method for detecting an interaction between a bait and a prey in a mammalian cell, comprising:
(a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey;
(b) incubating the cell for 30 hours or less; and
(c) detecting the location of the fluorescent polypeptide signal, if present surrounding the cell membrane, indicating an interaction between the bait and the prey.

22. A method of identifying an agent that disrupts interaction between a bait and a prey, comprising:
(a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey;
(b) contacting the mammalian cell with a test agent;
(c) incubating the cell for 30 hours or less; and
(d) detecting a decrease of the fluorescent polypeptide signal surrounding the cell membrane compared to the level of the fluorescent polypeptide signal surrounding the cell membrane in the absent of the test agent, if present, thereby detecting an agent that disrupts an interaction between the bait and the prey.

23. A method of identifying an agent able to enhance interaction between a bait and a prey, including:
(a) providing a mammalian cell containing: (i) a bait nucleotide sequence encoding a bait fusion protein, including a fluorescent polypeptide and a bait; (ii) a prey nucleotide sequence encoding a prey fusion protein, including a membrane-localizing signal and a prey;
(b) contacting the mammalian cell with a test agent;
(c) incubating the cell for 30 hours or less; and
(d) detecting a increase of the fluorescent polypeptide signal surrounding the cell membrane compared to the level of the fluorescent polypeptide signal surrounding the cell membrane in the absent of the test agent, if present, thereby detecting an agent able to enhance an interaction between the bait and the prey.
